SEQUENCE LISTING (14 sequences)

SEQ ID NO: 1 - DNA, 4377 bp, Homo sapiens
  gene (1)..(4377)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgagttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagttgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 2 - protein, 1458 aa, Homo sapiens
  PEPTIDE (1)..(1458)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEVGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 3 - DNA, 4377 bp, Homo sapiens
  gene (1)..(4377)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 4 - protein, 1458 aa, Homo sapiens
  PEPTIDE (1)..(1458)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 5 - DNA, 40 bp, Artificial Sequence
  primer_bind (1)..(40): Synthesized forward primer for F309S mutation
ccttggacagtttctactgagttgtcatatctcttcccac

SEQ ID NO: 6 - DNA, 40 bp, Artificial Sequence
  primer_bind (1)..(40): Synthesized reverse primer for F309S mutation
gtgggaagagatatgacaactcagtagaaactgtccaagg

SEQ ID NO: 7 - DNA, 31 bp, Artificial Sequence
  primer_bind (1)..(31): Synthesized forward primer for D519V mutation
cagtgactgtagaagttgggccaactaaatc

SEQ ID NO: 8 - DNA, 31 bp, Artificial Sequence
  primer_bind (1)..(31): Synthesized reverse primer for D519V mutation
gatttagttggcccaacttctacagtcactg

SEQ ID NO: 9 - DNA, 4377 bp, Homo sapiens
  gene (1)..(4377)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgagttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 10 - protein, 1458 aa, Homo sapiens
  PEPTIDE (1)..(1458)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 11 - DNA, 4377 bp, Homo sapiens
  gene (1)..(4377)
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagttgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 12 - protein, 1458 aa, Homo sapiens
  PEPTIDE (1)..(1458)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEVGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 13 - DNA, 4320 bp, Homo sapiens
  gene (1)..(4320)
gccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgagttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagttgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccagcaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcactgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 14 - protein, 1439 aa, Homo sapiens
  PEPTIDE (1)..(1439)
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLSCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEVGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

(19) United States
(12) Patent Application Publication
(10) Pub. No.: US 2015/0337028 A1
(43) Pub. Date: Nov. 26, 2015

(54) Double Mutant Coagulation Factor VIII and Methods Thereof

(71) Applicants: Centre for Bioseparation Technology-VIT, Vellore (IN); Christian Medical College, Vellore (IN)

(72) Inventors: Vijayalakshmi Mookambeswaran ARUNACHALAM, Vellore (IN); Bilgimol Chuvappumkal JOSEPH, Vellore (IN); Satheeshkumar Padikkara KUTTY, Vellore (IN); Sukesh Chandran NAIR, Vellore (IN); Alok SRIVASTAVA, Vellore (IN)

(21) Appl. No.: 14/427,936

(22) PCT Filed: Sep. 12, 2013
(86) PCT No.: PCT/IB2013/058490

§ 371 Date: Mar. 12, 2015

(30) Foreign Application Priority Data
Sep. 12, 2012 (IN) 3778/CHE/2012

(51) Int. Cl. C07K 14/755

(52) U.S. Cl. CPC C07K 14/755; A61K 38/00

(57) ABSTRACT

A double mutant B-domain deleted Factor VIII gene having mutations at Phe309Ser and Asp519Val, respectively, is disclosed for use in the field of haemophilia therapeutics. The disclosure a double mutant B-domain deleted Factor VIII protein having mutations at Phe309Ser and Asp519Val respectively, is also disclosed, as well as methods of producing the same. The B-domain deleted Factor VIII protein having mutations at Phe309Ser and Asp519Val shows enhanced activity and stability and therefore is used in the management of haemophilia.

### CROSS REFERENCE TO RELATED APPLICATIONS

The present application is a national stage filing of PCT Patent Application No. WO 2014/041500 A2, filed on Sep. 12, 2013, and entitled DOUBLE MUTANT COAGULATION FACTOR VIII AND METHODS THEREOF, which claims priority to Indian patent application serial number 3778/CHE/2012 filed on Sep. 12, 2012, the entire contents of which are incorporated by reference.

### FIELD OF THE INVENTION

The present disclosure is in the field of haemophilia therapeutics, particularly recombinant Factor VIII protein products. The present disclosure relates specifically to an expression cassette comprising a nucleotide sequence coding for a double mutant B-domain deleted Factor VIII gene having mutations at Phe309Ser and Asp519Val respectively. The disclosure also provides vectors and host cells transformed by the said expression cassette and corresponding methods thereof. The disclosure also relates to the production of a double mutant B-domain deleted Factor VIII protein having mutations at Phe309Ser and Asp519Val respectively. The protein shows enhanced activity and stability and therefore is used in the management of haemophilia.

### BACKGROUND OF THE INVENTION

Hemophilia A is a congenital or acquired disorder of coagulation that usually involves quantitative or functional disorder of a single coagulation protein. The available treatment for hemophilia A is the administration of plasma derived factor VIII or the infusion of recombinant factor VIII.

Factor VIII (FVIII) has 26 exons, encoding a polypeptide chain of 2351 amino acids (19 amino acids signal peptide and 2332 amino acids mature protein). It is a large multimeric protein with heavy (A1-A2-B domains) and light chains (A3-C1-C2 domains). The domain structure of factor VIII is arranged in order as (NH2)-A1-a1-A2-a2-B-a3-A3-C1-C2-(COOH) (a1, a2 and a3 indicate the spacers containing clusters of acidic amino acids). The molecular weight of a heavy chain spans between 90 and 210 kDa due to the limited processing of several proteolytic sites in the B domain. The molecular weight of a light chain is 80 kDa, as this is not processed (Kaufman et al, 1988, Bovenschen et al, 2005). Heavy and light chains remain associated with each other with the non-covalent linkage of a metal ion. (Vehar et al, 1984, Fass et al, 1982, Fay et al, 1986, Fay et al, 1992).

The expression level of factor VIII in heterologous systems is lower than that of other similar proteins expressed (Kaufman, et al. 1997). The reasons behind the limited expression of factor VIII in heterologous systems are: inefficient expression of factor VIII mRNA, ineffective transport of translated product from endoplasmic reticulum to the Golgi apparatus, interaction of misfolded proteins with chaperons in the ER etc.

Marquette et al. identified a 110 amino acid region within the A1 domain of factor VIII which inhibited factor VIII secretion from the ER (Marquette, et al. 1995). Further to this, the structural analysis revealed the presence of a hydrophobic β-sheet within this region (Swaroop, et al. 1997). Measures were taken to reduce the interaction of factor VIII with immunoglobulin-binding protein (BiP) by mutating the potential amino acids to hydrophilic amino acids (Swaroop, et al. 1997).

In the prior art, different mutations (single, double and triple) are reported to improve a single feature/trait. The mutations generated are aimed to improve the stability of the recombinant Factor VIII molecule by single [single mutations at the sites 519, 665 (A2 domain), and 1984 (A3 domain)], double [eleven double mutations in different combinations at the sites of 519, 665, and 1984] and triple [three triple mutations at the sites of 519, 665, and 1984 sites]. Further, the prior art also deals with the mutations aiming to improve the secretion of recombinant Factor VIII and concerned only about the amino acids in the A1 domain which interact with the proteins in the ER secretory pathway. The mutations generated are single [nine single mutations at the positions 293, 294, 300, 309, 310, 306, and 299 (present in the 110 amino acid stretch in A1 domain which interacts with chaperons)], double [five double mutations with different combinations of double mutations at the positions 293, 294, 300, 309, 310, 306, and 299], and triple mutations.

The point ‘synergism’ is applicable only if the mutations considered are pertaining to a single character. The activity of the recombinant Factor VIII is ultimately decided by the amount of protein successfully secreted with significant stability. The prior art so far has not reported any studies which tried to combine the two features—secretion and stability. Thus, the present disclosure aims at overcoming the drawbacks of the prior art by disclosing a double mutant showing improved activity due to the combined features of increased stability and secretion.

### SUMMARY OF THE INVENTION

Accordingly, the present disclosure relates to a nucleotide sequence set forth as SEQ ID No. 1 or a nucleotide sequence comprising the sequence set forth as SEQ ID No.1; an amino acid sequence set forth as SEQ ID No. 2 or an amino acid sequence comprising the sequence set forth as SEQ ID No.2; an expression cassette comprising nucleotide sequence set forth as SEQ ID No. 1 or a nucleotide sequence comprising the sequence set forth as SEQ ID No.1; a vector having an expression cassette of the present disclosure; a host cell comprising the vector of the present disclosure; a method of obtaining a nucleotide sequence set forth as SEQ ID No. 1 or its corresponding amino acid sequence, said method comprising the act of mutating a nucleotide sequence set forth as SEQ ID NO. 3 at positions 925-927 and 1555-1557, or mutating a corresponding amino acid sequence set forth as SEQ ID NO. 4 at positions 309 and 519; a method of obtaining a transformed host cell comprising an expression cassette of the present disclosure, said method comprising the acts of inserting the expression cassette of the present disclosure into a vector; and transforming the host cell with said vector to obtain the transformed host cell; a nucleotide sequence as set forth in SEQ ID No.13; an amino acid sequence as set forth in SEQ ID NO. 14; a protein having the amino acid sequence set forth as SEQ ID No. 14; a method of producing a protein having the amino acid sequence set forth as SEQ ID No. 14, said method comprising acts of mutating 925-927 and 1555-1557 positions of the nucleotide sequence set forth as SEQ ID No. 3 to obtain a nucleotide sequence set forth as SEQ ID No. 1; transforming a host cell comprising vector having an expression cassette comprising the nucleotide sequence set forth as SEQ ID No. 1; and culturing the transformed host cell in a culture medium for producing the said protein; a composition comprising a protein having an amino acid sequence set forth as SEQ ID No. 14 optionally along with excipients; a kit comprising mature recombinant double mutant factor VIII protein having an amino acid sequence set forth as SEQ ID No. 14; a method of enhancing plasma that is deficient in factor VIII, said method comprising the act of addition of mature recombinant double mutant factor VIII protein having an amino acid sequence set forth as SEQ ID No. 14 to the plasma that is deficient in factor VIII; a method of activating factor X, said method comprising the act of incubating factor X, or a sample comprising factor X, with mature recombinant double mutant factor VIII protein having an amino acid sequence set forth as SEQ ID No. 14 to activate the factor X; and a method of managing coagulation disorders, said method comprising the act of administering to a subject in need thereof, a composition comprising a protein having an amino acid sequence set forth as SEQ ID No. 14, optionally along with a pharmaceutically acceptable carrier, or excipient, or a combination thereof.

### BRIEF DESCRIPTION OF THE DRAWINGS

In order that the invention may be readily understood and put into practical effect, reference will now be made to exemplary embodiments as illustrated with reference to the accompanying figures. The figures together with a detailed description below, are incorporated in and form part of the specification, and serve to further illustrate the various embodiments, principles and advantages, in accordance with the present disclosure where:

FIG. 1A is an image depicting a mutation carried out at a site of the BDD recombinant Factor VIII gene;

FIG. 1B is an image depicting a mutation carried out at a site of the BDD recombinant Factor VIII gene;

FIG. 1C is an image depicting the mutations of FIG. 1A and FIG. 1B carried out at sites of the BDD recombinant Factor VIII gene;

FIG. 2 is a photograph of a gel depicting a western blot confirmation of wild-type Factor VIII (represented as BDD in the figure) and mutated recombinant Factor VIII (DM1 and DM2);

FIG. 3A is a graphical image depicting purification of recombinant ΔBDD-FVIII using histidine ligand affinity chromatography;

FIG. 3B a photograph of a gel depicting an SDS-PAGE analysis of the purified fractions of FIG. 3A;

FIG. 4 is an image depicting a pcDNA 3.1 vector construct; and

FIG. 5 is a photograph of a gel depicting a western blot analysis of purified fractions.

### DETAILED DESCRIPTION OF THE PREFERRED EMBODIMENTS

The present disclosure relates to a nucleotide sequence set forth as SEQ ID NO. 1, or a nucleotide sequence comprising a sequence set forth as SEQ ID NO.1.

In an embodiment of the present disclosure, the nucleotide sequence of SEQ ID NO. 1 corresponds to a B-domain deleted Factor VIII gene and comprises mutations at the 925-927 and 1555-1557 positions of said nucleotide sequence. The mutation in the nucleotide sequence at the position 925-927 corresponds to the replacement of phenylalanine with serine at the 309 position in the corresponding amino acid sequence, and the mutation in the nucleotide sequence at the position 1555-1557 corresponds to the replacement of aspartic acid with valine at the 519 position in the corresponding amino acid sequence.

In an embodiment of the present disclosure, the mutations are point mutations at the 925, 926 and 1556 positions of said nucleotide sequence.

The present disclosure also relates to an amino acid sequence set forth as SEQ ID NO. 2, or an amino acid sequence comprising sequence set forth as SEQ ID NO. 2.

In an embodiment of the present disclosure, the amino acid is corresponding to the nucleotide sequence of SEQ ID NO. 1.

In an embodiment of the present disclosure, the amino acid sequence corresponds to the B-domain deleted Factor VIII protein and comprises mutations at 309 and 519 positions.

The present disclosure also relates to an expression cassette comprising a nucleotide sequence set forth as SEQ ID NO. 1, or a nucleotide sequence comprising a sequence set forth as SEQ ID NO. 1.

In an embodiment of the present disclosure, the nucleotide sequence corresponds to B-domain deleted Factor VIII gene and comprises mutations at the 925-927 and 1555-1557 positions of said nucleotide sequence. The mutation in the nucleotide sequence at the position 925-927 corresponds to the replacement of phenylalanine with serine at the 309 position in the corresponding amino acid sequence, and the mutation in the nucleotide sequence at the position 1555-1557 corresponds to the replacement of aspartic acid with valine at the 519 position in the corresponding amino acid sequence.

The present disclosure also relates to a vector having an expression cassette of the present disclosure.

In an embodiment of the present disclosure, the vector comprising SEQ ID NO. 1 is inserted into an E. coli deposited under accession number MTCC 5855.

The present disclosure also relates to a host cell comprising a vector of the present disclosure.

The present disclosure also relates to a method of obtaining a nucleotide sequence set forth as SEQ ID NO. 1 or its corresponding amino acid sequence. The method comprises the act of mutating a nucleotide sequence set forth as SEQ ID NO. 3 at positions 925-927 and 1555-1557, or mutating a corresponding amino acid sequence set forth as SEQ ID NO. 4 at positions 309 and 519.

In an embodiment of the present disclosure, the nucleotide sequence set forth as SEQ ID NO. 3 corresponds to wild-type B-domain deleted Factor VIII gene, wherein the corresponding amino acid sequence set forth as SEQ ID NO. 4 corresponds to the corresponding wild type B-domain deleted Factor VIII protein.

In an embodiment of the present disclosure, the mutation in the nucleotide sequence at position 925-927 corresponds to the replacement of phenylalanine with serine at the 309 position in the corresponding amino acid sequence; and the mutation in the nucleotide sequence at position 1555-1557 corresponds to the replacement of aspartic acid with valine at the 519 position in the corresponding amino acid sequence

The present disclosure also relates to a method of obtaining a transformed host cell comprising an expression cassette or a vector comprising a nucleotide sequence set forth as SEQ ID NO. 1 or a nucleotide sequence comprising a sequence set forth as SEQ ID NO. 1. The method comprises the acts of inserting the expression cassette into a vector; and transforming the host cell with said vector to obtain the transformed host cell.

In an embodiment of the present disclosure, the host cell is a CHO cell.

The present disclosure also relates to a nucleotide sequence as set forth in SEQ ID NO.13.

In an embodiment of the present disclosure, the sequence corresponds to B-domain deleted Factor VIII gene and comprises mutations at 925-927 and 1555-1557 positions of said nucleotide sequence; wherein the mutation in the nucleotide sequence at the position 925-927 corresponds to the replacement of phenylalanine with serine at the 309 position in the corresponding amino acid sequence and the mutation in the nucleotide sequence at the position 1555-1557 corresponds to the replacement of aspartic acid with valine at the 519 position in the corresponding amino acid sequence.

The present disclosure also relates to an amino acid sequence as set forth in SEQ ID NO. 14.

In an embodiment of the present disclosure, the amino acid sequence corresponds to the mature BDD FVIII double mutant peptide.

In an embodiment of the present disclosure, the amino acid sequence corresponds to the mature BDD Factor VIII mutant peptide and comprises mutations F309S and D519V.

The present disclosure also relates to a protein having an amino acid sequence set forth as SEQ ID No. 14.

In an embodiment of the present disclosure, the amino acid sequence set forth as SEQ ID No. 14 corresponds to a mature BDD Factor VIII protein and comprises mutations F309S and D519V.

The present disclosure also relates to a method of producing a protein having an amino acid sequence set forth as SEQ ID No. 14. The method comprises the acts of mutating 925-927 and 1555-1557 positions of a nucleotide sequence set forth as SEQ ID No. 3 to obtain a nucleotide sequence set forth as SEQ ID No. 1; transforming a host cell comprising a vector having an expression cassette comprising the nucleotide sequence set forth as SEQ ID No. 1; and culturing the transformed host cell in a culture medium for producing the said protein.

In an embodiment of the present disclosure, the amino acid sequence set forth as SEQ ID No. 14 corresponds to mature B-domain deleted Factor VIII protein and comprises mutations F309S and D519V.

The present disclosure also relates to a composition comprising a protein having an amino acid sequence set forth as SEQ ID No. 14, optionally along with excipients.

In an embodiment of the present disclosure, the composition further comprise divalent metal ion(s).

In an embodiment of the present disclosure, the divalent metal ion(s) is, but not limited to, preferably Ca²⁺ ions.

In an embodiment of the present disclosure, the composition further comprises coagulation factors.

In an embodiment of the present disclosure, the amino acid sequence comprises mutations F309S and D519V.

The present invention also relates to a kit comprising a mature recombinant double mutant factor VIII protein having an amino acid sequence set forth as SEQ ID No. 14;

The present invention also relates to a method of enhancing plasma that is deficient in factor VIII, said method comprising act of adding a of mature recombinant double mutant factor VIII protein having amino acid sequence set forth as SEQ ID No. 14 to the plasma that is deficient in factor VIII;

The present invention also relates to a method of activating factor X. The method comprises the act of incubating factor X, or a sample comprising factor X, with a mature recombinant double mutant factor VIII protein having an amino acid sequence set forth as SEQ ID No. 14 to activate the factor X;

The present disclosure also relates to a method of managing coagulation disorders. The method comprises the act of administering to a subject in need thereof, a composition comprising a protein having an amino acid sequence set forth as SEQ ID No. 14, optionally along with pharmaceutically acceptable carrier or excipient or a combination thereof.

In an embodiment of the present invention, the coagulation disorder is, but not limited to heamophilia.

The present disclosure also relates to a double mutant recombinant Factor VIII.

In a preferred embodiment, the mutations in the recombinant Factor VIII are at two amino acid positions Phe309Ser (A1 domain) and Asp519Val (A2 domain), respectively.

As used herein, the following sequences are set forth and followed in the present disclosure—

SEQ ID NO. 1 (Total Length—4377 nucleotides): Nucleotide Sequence coding for the double mutant recombinant BDD-Factor VIII having mutations at 925-927 and 1555-1557 positions respectively. The nucleotide sequence contains 1-57 short signal peptide coding sequence.

The mutations of SEQ ID NO. 1 are at position 925-927 and at position 1555-1557, wherein these positions are calculated taking into account the short signal peptide coding sequence of 57 nucleotides at the beginning of SEQ ID NO. 1.

SEQ ID NO. 2 (Total Length—1458 amino acids): Amino acid sequence of the double mutant recombinant BDD-Factor VIII having mutations at Phe309Ser (A1 domain) and Asp519Val (A2 domain), respectively. The amino acid sequence contains 1-19 short signal peptide sequence.

The mutations of SEQ ID NO. 2 are at position 309 and at position 519, wherein these positions are calculated taking into account the short signal peptide coding sequence of 19 amino acid residues at the beginning of SEQ ID NO. 2.

SEQ ID NO. 3 (Total Length—4377 nucleotides): Nucleotide Sequence coding for the wild type BDD-recombinant Factor VIII. The nucleotide sequence contains 1-57 short signal peptide coding sequence.

SEQ ID NO. 4 (Total Length—1458 amino acids): Amino acid sequence of wild type BDD-recombinant Factor VIII. The amino acid sequence contains 1-19 short signal peptide sequence.

SEQ ID NO. 5: Forward primer for F309S mutation.

SEQ ID NO. 6: Reverse primer for F309S mutation.

SEQ ID NO. 7: Forward primer for D519V mutation.

SEQ ID NO. 8: Reverse primer for D519V mutation.

SEQ ID NO. 9: (Total Length—4377 nucleotides): Nucleotide Sequence of the single mutant recombinant BDD-Factor VIII having mutation at Phe309Ser (A1 domain). The nucleotide sequence contains 1-57 short signal peptide coding sequence.

SEQ ID NO. 10: (Total Length—1458 amino acids): Amino acid sequence of the single mutant recombinant BDD-Factor VIII having mutation at Phe309Ser (A1 domain). The amino acid sequence contains 1-19 short signal peptide sequence.

SEQ ID NO. 11: (Total Length—4377 nucleotides): Nucleotide Sequence of the single mutant recombinant BDD-Factor VIII having mutation at Asp519Val (A2 domain). The nucleotide sequence contains 1-57 short signal peptide coding sequence.

SEQ ID NO. 12: (Total Length—1458 amino acids): Amino acid sequence of the single mutant recombinant BDD-Factor VIII having mutation at Asp519Val (A2 domain). The amino acid sequence contains 1-19 short signal peptide sequence.

SEQ ID NO. 13: (Total length 4320): Nucleotide sequence coding for the mature double mutant recombinant BDD-Factor VIII peptide having mutations at 925-927 and 1555-1557 positions, respectively.

SEQ ID NO. 14: (Total length 1439): Amino acid sequence coding for the mature peptide for the double mutant recombinant BDD-factor VIII having mutations F309S and D519V.

In an embodiment of the present disclosure, the nucleotide sequence as set forth in SEQ ID No. 13 codes for the mature double mutant recombinant BDD-Factor VIII having mutations at 925-927 and 1555-1557. Further, the amino acid sequence as set forth in SEQ ID NO. 14 is the mature double mutant recombinant BDD Factor VIII having mutations F309S and D519V.

In an embodiment of the present disclosure, the nucleotide sequence as set forth in SEQ ID No. 1 codes for the double mutant recombinant BDD-Factor VIII containing mutations at 925-927 and 1555-1557 which also includes the sequence for the signal peptide.

Further, the amino acid sequence as set forth in SEQ ID NO. 2 is the double mutant recombinant BDD Factor VIII having mutations F309S and D519V including the amino acid sequence of the signal peptide.

As used herein, “management” or “managing” refers to preventing a disease or disorder from occurring in a subject, decreasing the risk of death due to a disease or disorder, delaying the onset of a disease or disorder, inhibiting the progression of a disease or disorder, partial or complete cure of a disease or disorder and/or adverse affect attributable to the said disease or disorder, obtaining a desired pharmacologic and/or physiologic effect (the effect may be prophylactic in terms of completely or partially preventing a disorder or disease or condition, or a symptom thereof and/or may be therapeutic in terms of a partial or complete cure for a disease or disorder and/or adverse affect attributable to the disease or disorder), relieving a disease or disorder (i.e. causing regression of the disease or disorder).

In an embodiment of the present disclosure, the double mutant recombinant Factor VIII showcases significantly improved secretion, increased circulating half life and stability and reduced immunogenicity.

In another embodiment of the present disclosure, the double mutant recombinant Factor VIII gene is expressed in mammalian heterologous expression systems, not limited to CHO, BHK, sf9, HEK 293 F and Hep3B cell lines. In a preferred embodiment, the double mutant recombinant Factor VIII gene is expressed in a CHO cell line.

In another embodiment of the present disclosure, the double mutant recombinant Factor VIII gene is expressed in a pichia expression system or a plant based expression system.

In an embodiment of the present disclosure, the double mutant recombinant Factor VIII gene is SEQ ID NO. 1.

In an embodiment of the present disclosure, the double mutant recombinant Factor VIII gene is SEQ ID NO. 13.

In an embodiment of the present disclosure, the double mutant recombinant Factor VIII gene set forth as SEQ ID NO. 13 is inserted into a vector having a sequence coding for signal peptide.

In an embodiment of the present disclosure, the double mutant recombinant Factor VIII of the present disclosure results in higher secretion, stability and activity. The double mutant F309S+D519V shows an increased activity when compared to the wild type, single mutant F309S and D519V. The increased activity of the double mutant is explained by the synergistic effect of this combination (F309S+D519V) over the individual mutations F309S and D519V alone, wherein the functional roles of amino acids at these positions improve the secretion, stability and activity.

In an embodiment of the present disclosure, addition of Calcium ion in the formulation/composition comprising double mutant recombinant factor VIII increases the unit activity of double mutant recombinant Factor VIII (ΔBDD-FVIII). The increased activity observed with Ca²⁺ is due to the effect of this specific combination of F309S and D519V mutations together in a single construct. Serine at 309 position is important in metal ion interaction over phenylalanine when the next position is occupied by Cysteine (Cys310). A mutation at 519 changes the conformational positioning of A1 and A3 and makes more amenable to Factor IXa binding through metal ion interaction. So putting this together, the double mutant of the present disclosure shows more stability compared to wild type BDD-FVIII. Further, reconstitution buffer combinations with calcium increases the activity of this double mutant.

Addition of divalent metal ions to the recombinant preparations used for therapeutic purposes is not a common practice. The structural elucidation of Factor VIII molecule, indicates that its interaction between different domains reveal that metal ions play a vital role in maintaining its functional integrity. It is shown that when heavy chain and light chain of Factor VIII reconstitutes in the presence of divalent metal ions, they regain coagulation activity. Nonetheless, there are no reports so far which show that the recombinant preparation of Factor VIII increases its activity by the addition of divalent cations.

In an embodiment of the present disclosure, the present disclosure utilizes the expected non-linked heavy chain and light chain in the preparations (as they are non-functional when separated) by adding 5 mM CaCl₂ to facilitate the inter chain linking of heavy and light chain to make them functionally active. The mutation in A2 domain (D519V) significantly improves the inter domain interaction through calcium.

Specific mutations at crucial sites of any protein can alter its biological function. With regard to the therapeutic protein produced using heterologous system, the particular technique of the present study is used extensively to generate mutant proteins. Even though single mutations have been produced earlier, the double mutant of the present disclosure is significantly better in terms of its activity and stability. Thus, the approach of the present disclosure is based mainly on the practical aspects, concerning with the stability and secretion of Factor VIII molecule produced from CHO.

The double mutant of the present invention is referred throughout the specification interchangeably as double mutant B-domain deleted Factor VIII, double mutant (Phe309Ser+Asp519Val), double mutant (F309S+D519V), double mutant BDD-FVIII, double mutant BDD-FVIII (Phe309Ser+Asp519Val), double mutant BDD-FVIII (F309S+D519V), double mutant recombinant factor VIII, recombinant BDD-FVIII-double mutant, rBDD-FVIII-DM and ΔBDD-FVIII. The expressed double mutant mature protein of the present invention contains the sequence as denoted by SEQ ID NO. 14, encoded by the nucleotide sequence of SEQ ID NO. 1.

The double mutant protein of the present invention containing the sequence as denoted by SEQ ID NO. 2 corresponds to the nucleotide sequence of SEQ ID NO. 1.

The present disclosure discloses the double mutant BDD-FVIII (Phe309Ser+Asp519Val) which has been deposited with an International Depository Authority as per the requirement under the Budapest treaty.

The cDNA construct in Escherichia coli-pcDNA for the recombinant BDD-FVIII-double mutant (rBDD-FVIII-DM) has been deposited with the International Depository, “The Microbial Type Culture Collection and Gene Bank (MTCC)” and has been accorded the accession number as MTCC 5855.

Additional embodiments and features of the present disclosure will be apparent to one of ordinary skill in art based upon the description provided herein. However, the examples and the figures should not be construed to limit the scope of the present disclosure.

### EXAMPLE 1

### Materials Used

E. coli strain DH5α is used for cloning and maintaining the plasmids. The wild type recombinant Factor VIII cDNA [pcDNA3.1(+) BDD-FVIII] expression construct is used. Mammalian cell line CHO is used as a heterologous protein expression system and is purchased from NCCS, Pune. CHO cells are cultured in DMEM-F12 medium containing 10% (v/v) fetal bovine serum (FBS) at 37° C. in a 5% CO₂ incubator. Commercially available purified recombinant Factor VIII is purchased from Epitomics (France). Polyclonal antibodies against C2 domain of Factor VIII are raised in house. Restriction endonucleases are purchased from New England Biolabs. DMEM-F12 and FBS are purchased from Himedia and Lipofectamine 2000 Reagent is purchased from Invitrogen. Serum free media for CHO cell line is purchased from Sigma. Geniticin is purchased from Invitrogen and Pfu is purchased from Fermentas. Sepharose 4B coupled with Histidine-Bisoxirine is used. Coametric instruments are used for chromogenic assay.

### EXAMPLE 2

### Construction of Mutated Recombinant Factor VIII

Site-directed mutagenesis of wild type recombinant Factor VIII cDNA (B domain deleted Factor VIII) is carried out. Mutations at F309S (Phe309Ser) and D519V (Asp519Val) are done using pfu polymerase with specific primers having desired mutation site integrated into it.

The pcDNA3.1(+) vector containing Factor VIII cDNA is used as template for site-directed mutagenesis. In an embodiment, the expression cassette is provided in FIG. 4—
Vector (pcDNA3.1)—5.4 Kb
Insert (pcDNA-FVIII)—4.6 Kb
Total plasmid size—10 Kb

The specific primers are designed to mutate BDD-FVIII using the program PrimerX (http://www.bioinformatics.org/primerx/). The GC content and melting temperature is checked by MBCF (Molecular biology core facilities) oligo calculator (http://mbcf.dfci.harvard.edu/docs/oligocalc.html). The PCR is performed using the following primers:

Primers for F309S-
Forward primer (SEQ ID NO. 5):
5′ CCTTGGACAGTTTCTACTGAGTTGTCATATCTCTTCCCAC 3′
Reverse primer (SEQ ID NO. 6):
5′ GTGGGAAGAGATATGACAACTCAGTAGAAACTGTCCAAGG 3′
Primers for D519V-
Forward primer (SEQ ID NO. 7):
5′ CAGTGACTGTAGAAGTTGGGCCA ACT AAATC 3′
Reverse primer (SEQ ID NO. 8):
5′ GATTTAGTTGGCCCAACTTCTACAGTCACTG 3′

The specific PCR Programme cycling parameters are as follows—
a. Initial denaturation for 4 minutes (94° C.) followed by 20 cycles of denaturation for 30 sec (94° C.);
b. Annealing at 64.5° C. for 45 sec;
c. Extension at 68° C. for 3 min followed by a single cycle of Final extension at 72° C. for 7 min.

The PCR amplicons obtained are further digested with Dpnl and transformed into DH5α competent cells. The positive clones are selected and sequenced for mutation integration confirmation.

### Results

The double mutant recombinant Factor VIII cDNA [pcDNA3.1(+)ΔBDD-FVIII] expression construct having mutations at Phe309Ser and Asp519Val is generated by site directed mutagenesis. Mutation integration is confirmed by DNA sequencing.

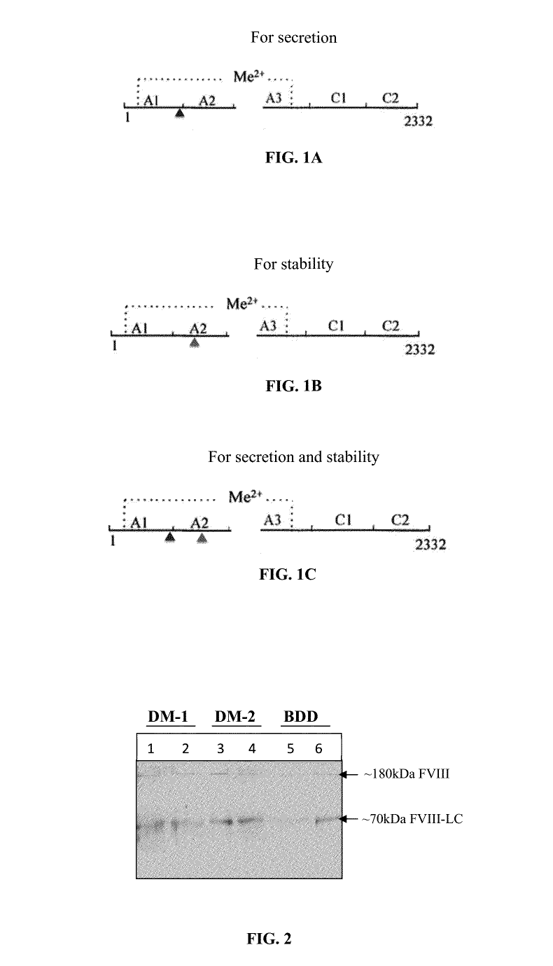

FIGS. 1A, 1B and 1C depict the mutations carried out at the various sites of the BDD recombinant Factor VIII gene.

### EXAMPLE 3

### Protein Expression Studies

Mammalian cell line CHO is cultured in 10% FBS. Once the cells attain 70-80% confluency, antibiotic free media is added to the cells and then transfected with wild type recombinant Factor VIII cDNA [pcDNA3.1(+) BDDFVIII] expression construct and double mutant recombinant Factor VIII cDNA [pcDNA3.1(+)ΔBDD-FVIII] expression construct in independent experiments respectively. Transfection is carried out using Lipofectamine reagent. After 6 hours of transfection, optimal media is changed and complete media is added. 48 hours post transfection, antibiotic Geniticin is added to the plate for selecting stable clones. Single stable clones are picked up from the plate and are transferred to 96 well plate containing complete media with 560 μg/ml geniticin. Sufficient passaging is provided to the cells before culturing them in 75 cm² flasks with 560 μg/ml geniticin. The cell culture supernatant is collected at various time points. For purification and further studies, stable clones are cultured in serum free media.

To analyze the expressed protein, Sodium Dodecyl Sulphate (SDS) poly acrylamide gel electrophoresis (PAGE) is carried out according to Laemmli and western blot is done to see the expression of the protein. Cell culture supernatant is mixed with 5×SDS sample loading buffer and boiled for 10 minutes. The samples are run on 8% SDS-PAGE gels at 80 volts for 120 minutes using Bio-Rad mini protein system. The resolved protein is transferred to nitrocellulose membrane at 90 volts 90 minutes in transfer buffer using Bio-Rad mini transblot apparatus. After transferring the protein to the nitrocellulose membrane, it is blocked with 5% skimmed milk powder. The membrane is then washed with PBS-T and incubated with anti C2 polyclonal antibody raised in-house for 2 hours and then with anti IgG conjugated with alkaline phosphatase. The positive reactivity is visualized using 5-bromo-4chloro-3-indolylphosphate/nitro blue tetrazonium (BCIP-NBT) as a substrate.

The aforementioned protein expression aspects are provided in a greater detail as follows:

### Materials

### Media Requirements

EX-CELL® 325 PF CHO serum-free medium for CHO Cells without L-glutamine, protein free, liquid, sterile-filtered, suitable for cell culture is purchased from Sigma Aldrich, Bangalore. Fetal bovine serum (FBS) and DMEM-F12, are purchased from Himedia laboratories, India.

### Antibiotics and Reagents

Trypsin and Penicillin-streptomycin solution are purchased from Himedia laboratories, India. Geniticin is purchased from Invitrogen, Lipofectamine™2000 is purchased from Invitrogen. Other chemicals are obtained from Sigma-Aldrich (USA), SRL (India) and Merck Limited (India). Pfu DNA polymerase and dNTPs are obtained from Fermentas. T4 DNA ligase and restriction enzymes are purchased from New England Biolabs, U.S.A. Antibiotics Kanamycin and Ampicillin are from Himedia Lab, India. Kits used for plasmid isolation and gel extraction are from Sigma Aldrich, USA. Other chemicals are obtained from SRL (India) and Merck Limited (India). The oligonucleotide primers are synthesized at Sigma Aldrich Ltd (Bangalore, India).

### Culture Wares

Petri dishes are purchased from Tarsons, 96 well plates, 24 well plates, 12 well plates, 6 well plates, and 25 cm² culture flasks are purchased from Nunclon, India. 75 cm² culture flasks are purchased from Himedia Laboratories, India. Cryo vials are purchased from Tarsons.

### Instruments

The instruments used in the present disclosure are Phase contrast microscope (Olympus, USA), Centrifuge (Hettich Zentrifugen, Germany), Deionized water system (Milli Q, Millipore), Freezers −20° C. and −80° C., CO₂ Incubator (Memmert, Germany), PCR machine (Eppendorf, Thermocycler model: 22331, Hamburg), Electrophoresis unit (Bio-Rad, USA), Gel documentation system (Bio rad, USA), Spectrophotometer (Beckman coulter, USA), Centrifuge (Eppendorf, Germany), Speed Vac, Deionized water system (Milli Q, Millipore), Ice machine, Incubators, Autoclave, Laminar air flow, Sonicator, pH meter, Water bath and minor lab equipments.

### Methods

### Media Preparation

DMEM-F12 powder—15-16 g; Autoclaved sterile water—1 L
Penicillin-Streptomycin solution—1-2 ml
Sodium bicarbonate—1-1.3 g

Milli Q water is autoclaved in a sterile flask. DMEM-F12 powder is added to sterile water and allowed to dissolve. 1.2 g of sodium bicarbonate is added to the media. 2 ml of penicillin-streptomycin antibiotics is added. pH of media is adjusted with NaOH or HCl. Media is filtered using 0.45μ filters. Sterility of the media is checked by overnight incubation at 37° C. in incubator with 5% CO₂. 10% FBS is added to sterile media for adherent cell culture.

### PBS Preparation

NaCl—137 mM
KCl—2.7 mM
Na₂HPO₄—4.3 mM
KH₂PO₄—1.47 mM

The solution is dispensed into aliquots and sterilized by autoclaving. The solution is stored at room temperature between 22-24° C.

### Culturing of CHO Cells

CHO Cells are kept in liquid nitrogen until they are ready to be thawed. To thaw the cells, cryo-vials are placed in water bath at 37° C. and cells are allowed to thaw. After homogenizing cells, it is transferred to a 15 ml tarson tube with 9 ml DMEM-F12 media supplemented with 10% FBS, centrifuged at 1500 rpm for 5 minutes. The supernatant is discarded after centrifugation and pellet is suspended in 1 ml of complete media and transferred it to 25 cm² culture flasks with 5 ml complete media. Culture flasks are placed in incubator at 37° C. supplied with 5% CO₂. The cells are allowed to adhere to the surface. Cell growth is observed every 24 hours under phase contrast microscope. Once the cell reached 90-100% confluency, the media is discarded and passaged.

### Transfection

### Requirements

CHO cell line
Lipofectamine
Plasmid DNA pcDNAFVIII mutated
DMEM-F12 media
FBS

### Procedure

One day before transfection, CHO cells are seeded onto 6 well plates. Each well has 0.5-2×10⁵ cells in 500 μl growth medium without antibiotics. Prior to transfection, the complexes are prepared separately. 4 μg of DNA is diluted in 50 μl of optimum medium (media without FBS). The mixture is gently mixed. 10 μl of lipofectamine 2000 is diluted in 50 μl optimum medium and is incubated for 5 minutes. After 5 minutes incubation, diluted DNA and diluted Lipofectamine 2000 are combined (total volume 100 μl). It is mixed gently and incubated for 20 minutes at room temperature. Medium is aspirated from plates and fresh medium is added without FBS and antibiotics before adding the complexes. 100 μl of complexes are added to each well containing cells and medium. It is mixed gently so that the complex is distributed evenly and incubated at 37° C. in CO₂ incubator. Complete medium is added after 6 hours of transfection. The cells are allowed to grow for 24 hours. Expression level is determined using GFP protein as control.

### Selection of Stable Clones Expressing Mutated Recombinant FVIII Protein (ΔBDD-FVIII) in CHO Cell Line

### Requirements

DMEM-F12 media
Pen-strep solution
Trypsin
FBS
Geniticin

### Procedure

After transfection, cells are allowed to grow under non-selective conditions for 24 hours. After 24 hours, selection media is added with 560 μg/ml of Geniticin. The cells are allowed to grow in selection media for 72 hours, after which fresh selection media is added. The clones are allowed to grow till it reached 90-100% confluency. Once the clones reached sufficient confluency, it is transferred to 96 well plate with same selection media. Clones are allowed to attach to surface and grow for 15 days or more. Passaging is done to 24 well plates, 12 well plates and 6 well plates with selection media.

### Selection of Single Clones Producing FVIII

Once the stable clones are produced, single clones are selected from stable clones. Clones are selected from 12 well plates. The cells are trypsinized and stable clones are diluted in 10 ml selection media. Dilution is made after cell counting. 100 μl of selection media statistically yields between 5-10 clones per 96 well plate, thereby minimizing the probability of wells with more than one clone. Cells are incubated under standard conditions and cells are feeded after 10-14 days with fresh selection medium. Clones are analyzed or further expanded as soon as cells in the non-transfected control wells have completely died. Once the resistant clones are identified, the cells are expanded and assayed for protein of interest by using appropriate analysis method.

### Culturing of Single Clones Expressing Mutated Factor VIII (ΔBDD-FVIII) in Serum Free Media

CHO cell line is first cultured in DMEM-F12 supplemented with 10% FBS. Concentration of serum is decreased stepwise in every other passage. Cells are finally maintained in lower concentrations of FBS, up to 0.5%. Selected mutated single clones are cultured in DMEM-F12 supplied with 0.5% of FBS and after two passages they are transferred to serum free media. 3×10⁵ cells/ml are transferred to serum free media and allowed to grow at 37° C. with 5% CO₂ supply. Cell culture supernatant is collected at various time points to check the protein production. Time points selected are 24, 48, 72, and 96 hours. Cell culture supernatant is then subjected to ELISA and Western blot for further analysis of secreted protein.

### Selection of Stable Clones Expressing Mutated BDD-FVIII (ΔBDD-FVIII) by ELISA

Stable CHO clones expressing mutated recombinant Factor VIII protein is analyzed by enzyme-linked immunosorbent assay (ELISA). Samples are collected from 96 well plate and coated onto ELISA plates for analysis. The basic principle of an ELISA is to use an enzyme to detect binding of antigen and antibody. Enzyme converts a colorless substrate to a colored product, indicating the presence of antigen:antibody complex.

### Requirements

Carbonate buffer/Bicarbonate buffer 0.1M, pH 9.6
NaHCO₃—8.4 g
Na₂CO₃—10.59 g
Milli Q water—1 L
pH adjusted to 9.6 and stored at room temperature
PBS pH 7.4
NaH₂PO₄—10 mM
Na₂HPO₄.2H₂O—10 mm
NaCl—150 mM
pH adjusted to 7.4

### Washing Buffer

PBS—1 L
Tween 20—0.05%
Tween 20 added and mixed on a magnetic stirrer

### Dilution Buffer

PBS—10 ml
Tween 20—0.05%
BSA—0.5%
BSA added to PBS with Tween 20 solution.

### Blocking Buffer

PBS—10 ml
BSA—0.5%

### TMB Solution

300 mg of TMB is dissolved in 100 ml of DMSO. Stored in dark

### H₂O₂

3% of H₂O₂ is prepared

### Citrate/Phosphate Buffer

Citric acid trisodium salt dihydrate—29.4 g
NaH₂PO₄.2H₂O—17.19 g
MilliQ water—1 L
The contents are added and pH adjusted to 5.0

### H₂SO₄

H₂SO₄—160 ml of concentrated H₂SO₄ is made up to 1 L with MilliQ water

### Procedure

CHO stable clones expressing Factor VIII protein is collected from 96 well plate. 50 μl of the sample is diluted in 0.1M bicarbonate buffer (pH 9.6), immobilized on 96 well ELISA plate for 20 hours at 4° C. Untransfected CHO is used as control. The plate is covered with aluminum foil. After 20 hours, plate is washed with washing solution, PBS and Tween 20. Remaining free space is blocked with BSA, incubating it for one hour at 37° C. It is then washed with washing buffer before adding primary antibody. Plate is incubated with polyclonal antibody raised against C2 domain of Factor VIII protein, with dilution of 1:10,000. Antibody is diluted in dilution buffer, incubated for an hour at 37° C. Finally incubated with secondary antibody, Anti-IgG-rabbit-HRP conjugated (Sigma Aldrich, Bangalore), with a dilution of 1:50,000 in dilution buffer. Incubation is done for an hour at 37° C. After washing thoroughly, 100 μl of TMB solution is added to the wells, incubated for 5 minutes. Reaction is stopped by adding 50 μl of H₂SO₄ (2M) to each well. Color change is noticed from blue to yellow. Reading is taken in an ELISA plate reader at 450 nm.

### Protein Analysis by SDS-PAGE

To analyze the expressed protein, SDS-PAGE is carried out according to Laemmli (Laemmli, 1970). Protein samples of 10 μl are mixed with 10 μl of 2×SDS sample loading buffer and boiled for 2 min. The samples are run on 10% SDS-PAGE gels at 100V for 90 min using Bio-Rad mini protein system (Bio-Rad Laboratories). The resolved protein samples are visualized by staining with Coomassie brilliant blue.

### Requirements

### Acrylamide/Bis-Acrylamide Solution (30.8%)

Acrylamide—30 gm
Bis-acrylamide—0.8 gm
In 100 ml of double distilled water

### Resolving Gel Buffer: (pH 8.8)

Tris base—18.18 gm in 70 ml of double distilled water
pH adjusted to 8.8 with conc. HCl
Water is added to make volume to 100 ml

### Stacking Gel Buffer (pH 6.8)

Tris base—7.6 gm
In 100 ml double distilled water
pH adjusted to 6.8 with conc. HCl
25 ml double distilled water to make 125 ml

### Sample Buffer

Stacking gel buffer—5 ml
10% SDS—8 ml
Glycerol—4 ml
β-mercaptoethanol—2 ml (added for reducing sample)
Bromo phenol blue indicator dye—2 mg
The volume made up to 20 ml by adding distilled water
Ammonium per Sulphate (10%)
TEMED solution
Butanol

### Tank Buffer (1×)

Tris—6.06 mg
Glycine—28.8 mg
SDS—1 gm
In Double distilled water—1000 ml

### Resolving Gel Composition (10%) (10 ml)

Acrylamide/bisacrylamide solution (30.8%)—3.3 ml
Double distilled water—4.0 ml
1.5M Tris (pH8.8)—2.5 ml
10% SDS—0.1 ml
TEMED—004 ml
10% APS—0.1 ml

### Stacking Gel Composition (5%) (3 ml)

Acrylamide/bisacrylamide solution (30.8%)—0.5 ml
Double distilled water—2.1 ml
1.5 M Tris (pH 6.8)—0.38 ml
10% SDS—0.03 ml
TEMED—0.003 ml
10% APS—0.03 ml

### Procedure

The two glass plates are assembled on a clean surface by keeping the longer glass plate containing spacers at the back and short thin plate in front. This glass plate sandwich is gently placed into the clamp assembly and tightened. This clamp assembly is placed on the casting stand, firmly against the rubber gasket at the base. The Resolving gel (8%) is prepared by mixing the appropriate concentration of the components gently, ensuring no air bubbles formation. The resolving gel is poured into the glass plate assembly. The gel is overlaid with butanol to ensure a flat surface and to exclude air. The butanol is washed off with water after the gel is set. Stacking gel (5%) is prepared and then poured on top of resolving gel. Comb (1 mm) is inserted gently and the gel is allowed to set. Then the comb is removed slowly. The gel assembly is placed in the buffer chamber and running buffer is added into the chamber. The sample is prepared by mixing the sample dye (2×) (reducing dye) with samples in the ratio of 1:1. Then the sample mixture is boiled for 5 min at 70° C., and centrifuged. 20 μl of the sample is loaded in the well and run at 100V for 90 min.

### Staining Procedure: Silver Staining

### Requirements

### Solution I

Acetone—30 ml
Water—30 ml
Trichloro acetic acid—0.75 g

### Solution II

Acetone—30 ml
Water—30 ml

### Solution III

Water—60 ml
Sodium thiosulphate 10%—25 μl
Formaldehyde—25 μl

### Solution IV

Silver nitrate—0.16 g
Water—60 ml

### Solution V

Water—60 ml
Sodium carbonate—1.2 g
Formaldehyde—100 μl

### Procedure

Gel is washed with water three times. The gel is fixed with Acetone and trichloro acetic acid for 5 minutes. After fixation, gel is washed with water three times. Then the gel is sensitized with sodium thiosulphate. After washing, gel is stained with silver nitrate by incubating in silver nitrate solution for 8 minutes. The gel is developed with developing solution, sodium carbonate. Once the bands are clear, the reaction is stopped by adding stop solution, glacial acetic acid. Gel is stored in water.

### Western Blotting

The expression of the recombinant protein is analyzed by transferring the protein from the SDS-PAGE gel to a nitrocellulose membrane and probing with anti Factor VIII polyclonal antibody raised in rabbit, in house.

### Requirements

### 10×PBS—1 L

NaCl—8 g
KCl—0.2 g
Na₂HPO₄—1.78 g
KH₂PO₄—0.24 g
Distilled water—Up to 1000 ml
pH—7.4

### Transfer Buffer—1 L

Tris—3.03 gm
Glycine—14.32 gm
Methanol—200 ml
Distilled water—800 ml
SDS (10%)—1 ml

### Washing Buffer

1×PBS—1 L
Tween 20—1 ml

### Blocking Buffer

Skimmed milk powder—5 gm
Tween 20—100 μl
1×PBS—100 ml
Primary antibody—Anti Factor VIII antibody (polyclonal, raised in house)
Secondary antibody—Alkaline phosphatase conjugated antibody
Substrate—BCIP/NBT tablets (Sigma)

### Procedure

SDS-PAGE gel is run at 100V, according to the procedure explained in section SDS-PAGE procedure section, till the blue dye front reaches the bottom of the gel. To transfer proteins from gel to nitrocellulose membrane, materials used for transfer are soaked in transfer buffer for 15 min. Then they are stacked in the following order; case (clear side), sponge, Whatman paper, membrane, gel, Whatman paper, sponge case (black side) and placed in the transfer apparatus with black side facing black. The apparatus is immersed into ice filled bucket to cool the transfer buffer. Transfer is carried out at 90V for 90 min using Bio-Rad electro transfer apparatus. After transfer, the membrane is stained with 1×Ponceau S for a min, the bands marked with pencil, destained in water and rinsed in 1×PBS. Then the membrane is blocked for 1 h in 50 ml of 1×PBS+5% non-fat dry milk+0.1% Tween 20 on a shaker. Then the membrane is incubated with 1:3000 dilution of primary antibody in blocking buffer and incubated at RT for 1 h or 4° C. overnight. The membrane is incubated with alkaline phosphatase conjugated secondary antibody (1:10,000 dilutions) in blocking buffer for 1 h at room temperature. Between each step the membrane is washed three times with wash buffer containing PBS and 0.1% Tween 20 (PBS-T). One tablet, dissolved in 10 ml of water, provides 10 ml of ready to use buffered substrate solution. The substrate solution contains BCIP (0.15 mg/ml), NBT (0.30 mg/ml), Tris buffer (100 mM), and MgCl2 (5 mM), pH 9.25-9.75. The membrane is incubated in the substrate solution for 5 min.

### Results

Transfection of CHO cell lines are carried out. Selection of clones producing Factor VIII protein is done using geniticin 560 μg/ml. Single clones are selected and maintained in 96 well plate, 48 well plate, 24 well plate, 6 well plate, 25 cm flask and 75 cm flask. Confirmation of the clones producing Factor VIII protein is done using ELISA (Table 1) and Western blot (FIG. 2). The clone confirmation by western blot is done using C2 polyclonal antibody raised in house.

TABLE 1

| S.NO | CELL LINE | ELISA VALUE |
|---|---|---|
| 1 | CHO | 0.176 |
| 2 | BDD FVIII | 0.304 |
| 3 | | 0.289 |
| 4 | | 0.305 |
| 5 | | 0.207 |
| 6 | | 0.311 |
| 7 | ΔBDD FVIII | 0.237 |
| 8 | | 0.307 |
| 9 | | 0.116 |
| 10 | | 0.328 |
| 11 | | 0.316 |

This table 1 depicts ELISA confirmation of clones producing recombinant Protein. The cell culture media collected after 72 hrs is used in the experiment. CHO (control), B Domain Deleted Factor VIII i.e. wild-type Factor VIII without mutation (BDD-FVIII), Mutated B domain Deleted Factor VIII 309S+519V i.e. Double mutant recombinant Factor VIII with mutations at F309S and D519V (ΔBDD-FVIII-309S+519V).

Clones given in bold indicate the high protein secreting clones.

The cell culture media collected after 48 and 72 hours is used in the experiment shown in FIG. 2. First four lanes (1-4) indicate samples collected for two clones of double mutant recombinant Factor VIII and lane 5 and 6 represent wild-type BDD-FVIII protein.

### Legend for FIG. 2:

Lane 1 and 2: protein expressed at 48 hours and 72 hours (DM-1)
Lane 3 and 4: protein expressed at 48 hours and 72 hours (DM-2)
Lane 5 and 6: protein expressed at 48 hours and 72 hours (wild-type BDD-FVIII without mutation). In FIG. 2, the arrows indicate the full length BDD-F VIII (^{˜}180 KDa) and Light chain of F VIII (^{˜}70 KDa). DM=Double mutant recombinant Factor VIII and BDD=B Domain Deleted wild-type Factor VIII without mutation.

### EXAMPLE 4

### Purification of Recombinant Factor VIII

The protein of interest (wild type and double mutant recombinant Factor VIII) is purified using histidine ligand affinity chromatography (HLAC). The column is packed with 1 mL of Histidine-Bisoxirine-Sepharose-4B gel. The column is equilibrated with binding buffer 20 mM Tris pH 6.0. Cell culture supernatant containing the desired protein is loaded to the column after adjusting the pH of the supernatant to pH6.0 at a flow rate of 2 ml per minute. After injecting the sample, all the non retained proteins are washed with the binding buffer till the baseline is achieved. The target protein is eluted with 20 mM Tris containing 0.1M Glycine, 0.03M Lysine and 0.3M CaCl₂ at pH 7.0. The eluted peaks are concentrated using Amicon filters and analyzed on SDS-PAGE and the protein concentration is calculated by Bradford method. The purified fraction is confirmed by western blot analysis.

Even though HLAC has been used successfully in the purification of a number of recombinant and native proteins, high value therapeutic proteins with high molecular weight such as recombinant Factor VIII has not been reported to be purified using this technique. This is the first report which shows the purification of recombinant Factor VIII from cell culture supernatant using HLAC.

The aforementioned affinity purification procedure is provided in detail as follows:

### Requirements

Column type: Sepharose 4B
Colume volume: 1 ml
Load: 2 mg of protein
Equilibration/binding buffer: 20 mM Tris pH6.0
Elution buffer: 20 mM Tris+0.1M Glycine+0.03M Lysine+0.3M CaCl₂ pH 7.0
Flow rate: 1 ml/min
Fraction volume: 2 ml

### Procedure

Cell culture supernatant containing BDD-FVIII and mutated BDD-FVIII protein (2 mg/50 ml) is loaded onto 1 ml Histidine-Bisoxirine-Sepharose 4B column, which is equilibrated with the equilibration/binding buffer at a flow rate of 1 ml/min. After injecting the sample, all the non retained proteins are washed with the binding buffer till the baseline is achieved. The target protein is eluted with elution buffer 20 mM Tris containing 0.1M Glycine, 0.03M Lysine and 0.3M CaCl₂. Protein concentration is determined by Bradford's assay. The purity of the eluted protein is analyzed by SDS-PAGE.

### Results

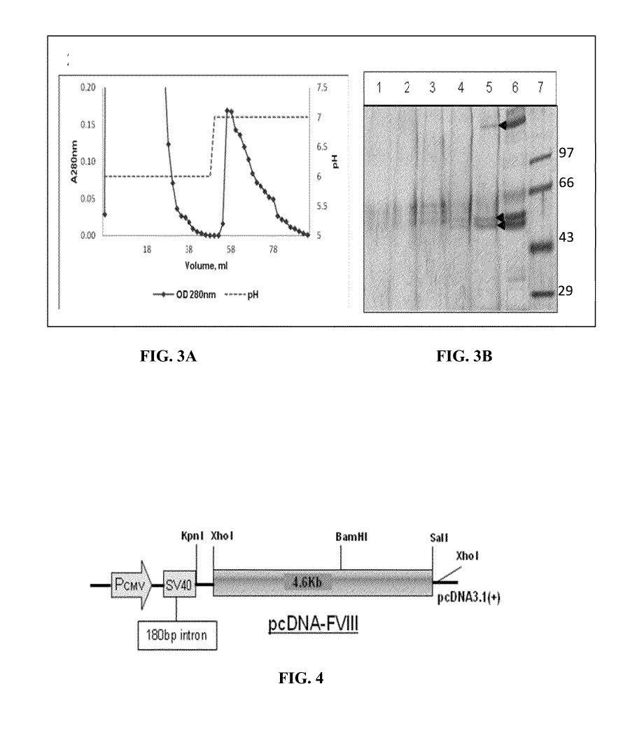

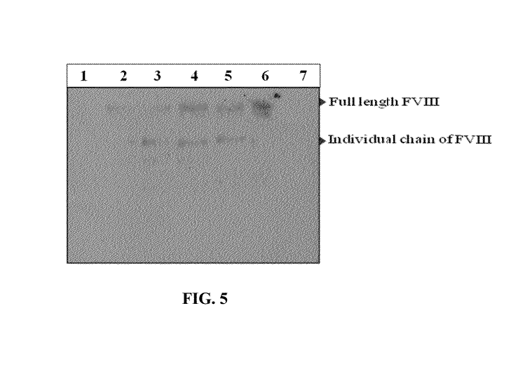

CHO cells are cultured in serum free media. Cell culture supernatant is collected after 48 hours. Cell culture supernatant containing mutated recombinant Factor VIII is subjected to affinity chromatography. Histidine-Bisoxirine-Sepharose-4B column is equilibrated with binding buffer 20 mM Tris pH 6.0. The pH of cell culture supernatant is adjusted to 6.0 and directly injected to the column after filtration with 0.22 μm filter. Flow through and elutions are collected for further analysis. The protein is eluted using 20 mM Tris+0.1M Glycine+0.03M Lysine+0.3M CaCl2 pH 7.0. The chromatogram shows a single peak (FIG. 3A). SDS-PAGE analysis shows a clear band corresponding to full length FVIII (FIG. 3B, Lane 5) which is confirmed by Western analsysis (FIG. 5). The double arrows in FIG. 3B correspond to fragments of FVIII which is also observed in purified commercial FVIII (Lane 6).

Thus, the chromatogram (FIG. 3A) shows a single peak with the elution buffer. Purified fractions are then subjected to SDS-PAGE analysis, as shown in FIG. 3B, wherein Lane 1=load, lane 2=flow through, lane 3=wash, lane 4=elution without concentrating, lane 5=elution after concentrating, lane 6=commercial Factor VIII, lane 7=medium range protein molecular weight markers. The arrows in FIG. 3B indicate the full length and individual chains of Factor VIII.

In FIG. 5, Lane 1=CHO, Lane 2=BDD-FVIII, Lane 3 & Lane 4=double mutant, Lane 5=commercial factor VIII, and Lane 6=D519V.

The heavy chain and light chain linkage of BDD-FVIII is stabilized only through metal ion. In the electrophoretic system, the electric current present will disrupt this linkage before the domain rearrangement happens. This explains the presence of the heavy chain and light chain separately due to the disruption in the linkage.

### EXAMPLE 5

### Activity Assays

To confirm the presence of Factor VIII, one stage clotting assay and chromogenic assays are performed.

### One Stage Clotting Assay

Factor VIII deficient plasma is dissolved in distilled or deionized water. Before use, it is allowed to stand for at least 15 minutes at 15 to 25° C., and then mixed carefully without foam formation. Wild type recombinant BDD Factor VIII and mutated BDD-FVIII sample produced in CHO cell lines are added to FVIII deficient plasma. APTT reagents are used according to the manufacturer's instructions. 0.025M of CaCl₂ is added to the mixture. The mixture is incubated at 37° C. for 2 minutes and the reading is taken in an automated coagulation analyzer. Normal clotting time is 30-40 seconds.

### Chromogenic Assay

For the photometric determination of Factor VIII activity, chromogenic assay is done. In the presence of Calcium and phospholipids, factor X is activated to factor Xa by factor IXa. This generation is stimulated by Factor VIII. The rate of activation of factor X solely depends on the amount of Factor VIII. Factor IXa hydrolyses the chromogenic substrate S-2765, thus liberating the chromophoric group, pNA. The color is then read photometrically at 405 nm. The generated factor Xa and thus the intensity of the color are directly proportional to the Factor VIII activity in the sample. The said chromogenic assay is further detailed as follows.

Appropriate amount of BDD-FVIII and mutated BDD-FVIII sample is taken in plastic test tubes. Specific controls for plasma or Factor VIII concentrates is calibrated against the international standard of Factor VIII. The detection limit is 0.05 IU/mL and low range of 0.005 IU/mL is detected in case of low range.

After reconstituting the Factor (Factor VIII, Factor IX, and Factor X) reagents, chromogenic substrate along with inhibitor is reconstituted and then the buffer is prepared.

### Results

The activity assays are carried out for double mutant BDD-FVIII Samples and wild type BDD-FVIII Samples. The purified Factor VIII samples are added to Factor VIII depleted plasma and one stage clotting assay is done (Table 2). Chromogenic assay result for wild type BDD-FVIII Samples and double mutant BDD-FVIII Samples reveal that the double mutant BDD-FVIII protein shows about 3.7 fold increase in activity compared to wild type BDD-FVIII protein (Table 2).

The one stage clotting assay and chromogenic assay was carried out for BDD-FVIII type, single and double mutant of the present invention and the results are tabulated below.

TABLE 2
One stage clotting assay results [the experiments are
repeated thrice and the mean values are represented]

| | % FVIII | Specific |
|---|---|---|
| Name | Activity | Activity (IU/mg) |
| BDD-FVIII | 0.795 | 2053 |
| BDD-FVIII (single mutant- | 0.823 | 4064 |
| Phe309Ser) | | |
| BDD-FVIII (single mutant- | 0.0091 | 24.82 |
| Asp519Val) | | |
| BDD-FVIII (double mutant) | 0.900 | 7978 |

### Inference

The Factor VIII double mutant has higher specific activity (3.88 fold) when compared to wild type BDD FVIII as determined by one stage clotting assay. The single mutant BDD-FVIII (Phe309Ser) shows approximately 2 fold increase when compared to the activity of the wild type BDD FVIII, whereas the single mutant BDD-FVIII (Asp519Val) had negligible activity.

TABLE 3
Chromogenic assay results

| | % of Factor | Units/Amount of Protein |
|---|---|---|
| Name | VIII activity | [Specific Activity] |
| BDD-FVIII | 0.419 | 1082 |
| BDD-FVIII (single mutant- | 0.432 | 2133 |
| Phe309Ser) | | |
| BDD-FVIII (single mutant- | 0.0009 | 2.482 |
| Asp519Val) | | |
| BDD-FVIII (double mutant) | 0.455 | 4033 |

### Inference

The Factor VIII double mutant has higher specific activity (3.727 fold) when compared to BDD FVIII as determined by chromogenic assay. The single mutant BDD-FVIII (Phe309Ser) shows approximately 2 fold increase when compared to the activity of the wild type BDD FVIII, whereas the single mutant BDD-FVIII (Asp519Val) had negligible activity.

### Conclusion

Both the one stage clotting assay and chromogenic assay results showed that there is synergistic effect shown by the double mutant which is reflected in terms of enhanced activity over the wild type BDD FVIII and any of the single mutant BDD-FVIII.

### EXAMPLE 6

### Addition of Divalent Metal Ion to the Purified Factor VIII Protein and Activity Assays

The purified wild type and double mutant Factor VIII samples are incubated with 5 mM CaCl₂ for 20 hours and the specific activity is measured using chromogenic assay.

### Results

Addition of divalent metal ion to the purified wild type and mutated Factor VIII protein results in an increased specific activity of the protein. The result of chromogenic assay indicates that there is a 6 fold increase in the specific activity of double mutant Factor VIII compared to wild type Factor VIII when CaCl₂ is added to both wild type and double mutant proteins and incubated for 20 hours (Table 4).

TABLE 4
One stage clotting assay results after
the addition of divalent metal ion

| | | Specific activity |
|---|---|---|
| | % FVIII | (Units/Amount of Protein) |
| Name | activity | [Specific Activity] |
| BDD-FVIII | 0.832 | 2148 |
| BDD-FVIII-F309S | 0.82 | 4049 |
| BDD-FVIII-D519V | 1.5 | 4092 |
| BDD-FVIII- | 1.46 | 12943 |
| F309S + D519V | | |

### EXAMPLE 8

### One Stage Clotting Assay Results of 10th, 20th and 30th Generation of the Clones of Wild Type and the Double Mutant FVIII

The one stage clotting assay results of the 10^{th} generation, 20^{th} generation and the 30^{th} generation of the clones of wild type BDD-FVIII was noted. The one stage clotting assay results of the 10^{th} generation, 20^{th} generation and the 30^{th} generation of the clones of the double mutant BDD-FVIII-F309S+D519V is tabulated in Table 6.

### Results

The results from the below table (Table 6) show the stability of % FVIII activity and the specific activity of the mutated factor VIII when compared to wild type factor VIII over 10^{th}, 20^{th} and the 30^{th} generations.

TABLE 6
One stage clotting assay results of 10th, 20th and 30th generation
of the clones of wild type and the double mutant FVIII

| | % FVIII | Specific |
|---|---|---|
| Name | activity | activity (IU/mg) |
| BDD-FVIII | 0.216 | 2160 |
| (10^{th} Generation) | | |
| BDD-FVIII | 0.207 | 2070 |
| (20^{th} Generation) | | |
| BDD-FVIII | 0.244 | 2440 |
| (30^{th} Generation) | | |
| BDD-FVIII-F309S + D519V | 0.874 | 7748 |
| (10^{th} Generation) | | |
| BDD-FVIII-F309S + D519V | 0.85 | 7727 |
| (20^{th} Generation) | | |
| BDD-FVIII-F309S + D519V | 0.88 | 7927 |
| (30^{th} Generation) | | |

1-35. (canceled)

36. An amino acid sequence as set forth in SEQ ID NO. 14.

37. The amino acid sequence as claimed in claim 36, wherein the amino acid sequence corresponds to a mature BDD FVIII double mutant peptide comprising mutations F309S and D519V.

38. The amino acid sequence as claimed in claim 36, the amino acid sequence having a signal peptide sequence as set forth in SEQ ID NO. 2.

39. A nucleotide sequence as set forth in SEQ ID No. 13.

40. The nucleotide sequence as claimed in claim 39, wherein the nucleotide sequence corresponds to a B-domain deleted Factor VIII gene and comprises at least one mutation at position 925-927 and at least one mutation at position 1555-1557 of said nucleotide sequence; wherein the at least one mutation in the nucleotide sequence at position 925-927 corresponds to the replacement of phenylalanine with serine at position 309 in a corresponding amino acid sequence, and further wherein the at least one mutation in the nucleotide sequence at position 1555-1557 corresponds to the replacement of aspartic acid with valine at position 519 in the corresponding amino acid sequence.

41. The nucleotide sequence as claimed in claim 40, wherein the at least one mutation at position 925-927 includes point mutations at position 925 and position 926 of said nucleotide sequence, and further wherein the at least one mutation at position 1555-1557 includes a point mutation at position 1556 of said nucleotide sequence.

42. The nucleotide sequence as claimed in claim 39, wherein the nucleotide sequence corresponds to an amino acid sequence as set forth in SEQ ID No. 14.

43. The nucleotide sequence as claimed in claim 39, the nucleotide sequence having a signal peptide coding sequence as set forth in SEQ ID NO. 1.

44. An expression cassette or a vector comprising a nucleotide sequence as claimed in claim 39.

45. The expression cassette as claimed in claim 44, wherein the expression cassette comprises SEQ ID NO. 13 along with a signal peptide coding sequence as set forth in SEQ ID NO. 1.

46. The expression cassette or vector as claimed in claim 44, wherein the vector is inserted into an E. coli deposited under accession number MTCC 5855.

47. A host cell comprising the vector as claimed in claim 44.

48. (canceled)

49. A method of obtaining a nucleotide sequence as set forth in SEQ ID No. 13 along with a signal peptide coding sequence of 1-57 nucleotides as set forth in SEQ ID NO. 1, said method comprising the steps of:
a. mutating a nucleotide sequence as set forth in SEQ ID NO. 3 at position 925-927 and position 1555-1557 after the signal peptide coding sequence of 1-57 nucleotides.

50. The method as claimed in claim 49, wherein the nucleotide sequence as set forth in SEQ ID No. 3 corresponds to a wild-type B-domain deleted Factor VIII gene and further wherein:
a. the mutation in the nucleotide sequence at position 925-927 after signal peptide coding sequence of 1-57 nucleotides corresponds to the replacement of phenylalanine with serine at position 309 in a corresponding amino acid sequence; and
b. the mutation in the nucleotide sequence at position 1555-1557 after signal peptide coding sequence of 1-57 nucleotides corresponds to the replacement of aspartic acid with valine at position 519-(delete space) in the corresponding amino acid sequence.

51. A method of obtaining a transformed host cell comprising an expression cassette as claimed in claim 44, said method comprising the steps of:
a. inserting the expression cassette into a vector; and
b. transforming a host cell with said vector to obtain the transformed host cell.

52. The method as claimed in claim 51, wherein the host cell is a CHO cell.

53. A method of producing a protein having an amino acid sequence as claimed in claim 36, said method comprising the steps of:
a. mutating at position 925-927 and position 1555-1557 after signal peptide coding sequence of 1-57 nucleotides of a nucleotide sequence as set forth in SEQ ID No. 3 to obtain a nucleotide sequence as set forth in SEQ ID NO.13 along with a signal peptide sequence as set forth in SEQ ID No. 1;
b. transforming a host cell comprising a vector having an expression cassette, the expression cassette comprising a nucleotide sequence as set forth in SEQ ID No. 1; and
c. culturing the transformed host cell in a culture medium for producing the protein.

54. The method as claimed in claim 53, wherein the amino acid sequence corresponds to a mature B-domain deleted Factor VIII protein and further wherein the amino acid sequence comprises mutations F309S and D519V.

55. A composition comprising a protein having an amino acid sequence as claimed in claim 36.

56. The composition as claimed in claim 55, wherein the composition includes excipients.

57. The composition as claimed in claim 55, wherein the amino acid sequence corresponds to a mature B-domain deleted Factor VIII protein and further comprises mutations F309S and D519V; and further wherein the composition includes divalent metal ions, wherein the composition is administered by a mode of administration selected from the group consisting of a parenteral mode, an intramuscular mode, an intravenous mode, a subcutaneous mode, and a combination mode thereof.

58. A kit comprising a mature recombinant double mutant factor VIII protein having an amino acid sequence as claimed in claim 36.

59. A method of enhancing plasma that is deficient in factor VIII, said method comprising the steps of:
a. adding a mature recombinant double mutant factor VIII protein having an amino acid sequence as claimed in claim 36 to the plasma.

60. A method of activating factor X, said method comprising the steps of:
a. incubating factor X or a sample comprising factor X with a mature recombinant double mutant factor VIII protein having an amino acid sequence as claimed in claim 36 to activate the factor X.

61. A method of managing a coagulation disorder, said method comprising the steps of:
a. administering to a subject in need thereof, a composition comprising a protein having an amino acid sequence as claimed in claim 36.

62. The method as claimed in claim 61, wherein the step of administering a composition to a subject further includes,
a. administering the composition along with one of a pharmaceutically acceptable carrier, an excipient, and a combination thereof.

63. The method as claimed in claim 61, wherein the coagulation disorder is haemophilia.

64. The method as claimed in claim 62, wherein the coagulation disorder is haemophilia.
